(19) United States
(12) Patent Application Publication
(10) Pub. No.: US 2013/0053647 A1
(43) Pub. Date: Feb. 28, 2013

(54) Operative Laryngoscope Dental Guard

(71) Applicant: Jason P. McChesney, Morgantown, WV (US)

(72) Inventor: Jason P. McChesney, Morgantown, WV (US)

(73) Assignee: WEST VIRGINIA UNIVERSITY, Morgantown, WV (US)

(21) Appl. No.: 13/592,441

(22) Filed: Aug. 23, 2012

Related U.S. Application Data
(60) Provisional application No. 61/575,574, filed on Aug. 24, 2011.

(51) Int. Cl. A61B 1/267

(52) U.S. Cl. 600/186

(57) ABSTRACT

A removable dental guard fitted to an operative laryngoscope is described to protect the dental structures of patients undergoing laryngoscopy procedures. The dental guard is made of resilient material and may be constructed of flame retardant material.

### CROSS REFERENCE TO RELATED APPLICATIONS

This patent application claims priority and benefit of provisional patent application having Ser. No. 61/575,574 filed on Aug. 24, 2011 and is incorporated in its entirety herein.

### FIELD OF THE INVENTION

This invention relates to a device and method for protecting dental structures of human patients undergoing laryngoscopy procedures with an operative laryngoscope.

### BACKGROUND OF THE INVENTION

Insertion and manipulation of conventional operative laryngoscope configurations are well known to be potentially damaging to the teeth of a patient undergoing laryngoscopy procedures. The teeth, particularly the anterior incisors, serve as a natural fulcrum for the laryngoscope as it is maneuvered laterally in the larynx of a patient. Dental guards similar to athletic mouth guards are quite often used to help reduce the potential for tooth or gum damage, however, these guards are not optimum since the guards do not always remain in place during a procedure and quite often need to be adjusted during critical phases of the medical procedure underway. An improved dental guard for operative laryngoscopy is needed.

### SUMMARY OF THE INVENTION

The instant invention greatly reduces the potential for dental damage caused by the rigid walls of an operative laryngoscope by employing a protective, removable sheath over the portion of the operative laryngoscope which may contact the patient's teeth during laryngoscopy procedures. The sheath is temporarily fixed (removable) to the operative laryngoscope in one embodiment of the invention and moves with the laryngoscope as medical personnel manipulate it in the patient's larynx thereby cushioning the dental surfaces which are used as a fulcrum or a lateral stop for the scope. The sheath is constructed of flexible, resilient material typically a few millimeters thick which can be easily mounted on the outer surface portion of operative laryngoscope that will extend through the patient's mouth during a laryngoscopy procedure. In one embodiment of the invention, the outer surface of the resilient material comprising the sheath is covered with a less resilient material to reduce sliding resistance against the patient's teeth.

### BRIEF DESCRIPTION OF THE DRAWINGS

FIG. 1 is a view of the operative laryngoscope inserted into the larynx through the oral cavity of a human patient with the dental guard sheath serving as a protective barrier between the teeth of the patient and the laryngoscope outer surfaces.

FIG. 2 is a view of the operative laryngoscope with the dental guard in place with the anchor strap fastened.

FIG. 3 is a view of a typical operative laryngoscope.

FIG. 4 is a view of the dental guard with the anchor strap fastened.

FIG. 5 is a view of the dental guard with the anchor strap unfastened.

FIG. 6 is a view of the dental guard with a less resilient outer surface included to reduce friction on dental surfaces.

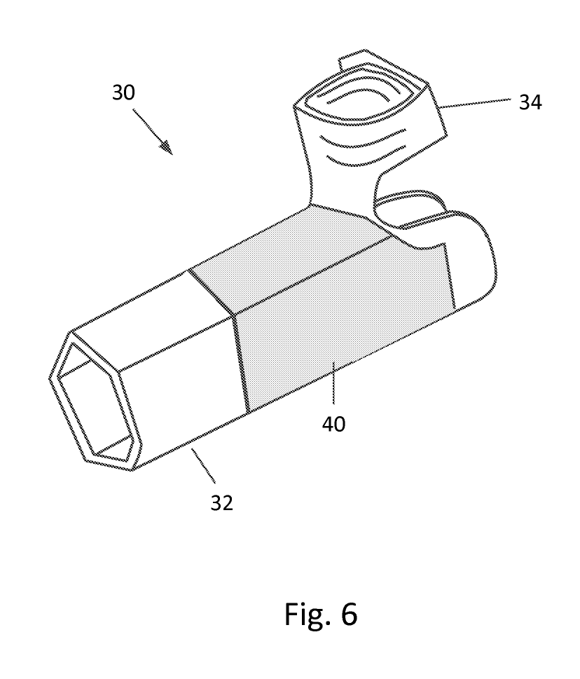

### DETAILED DESCRIPTION OF THE INVENTION

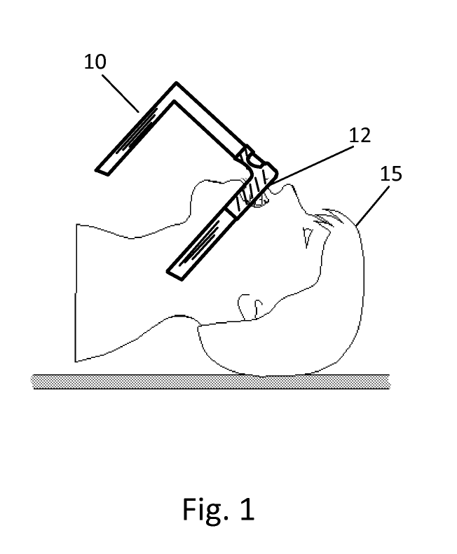

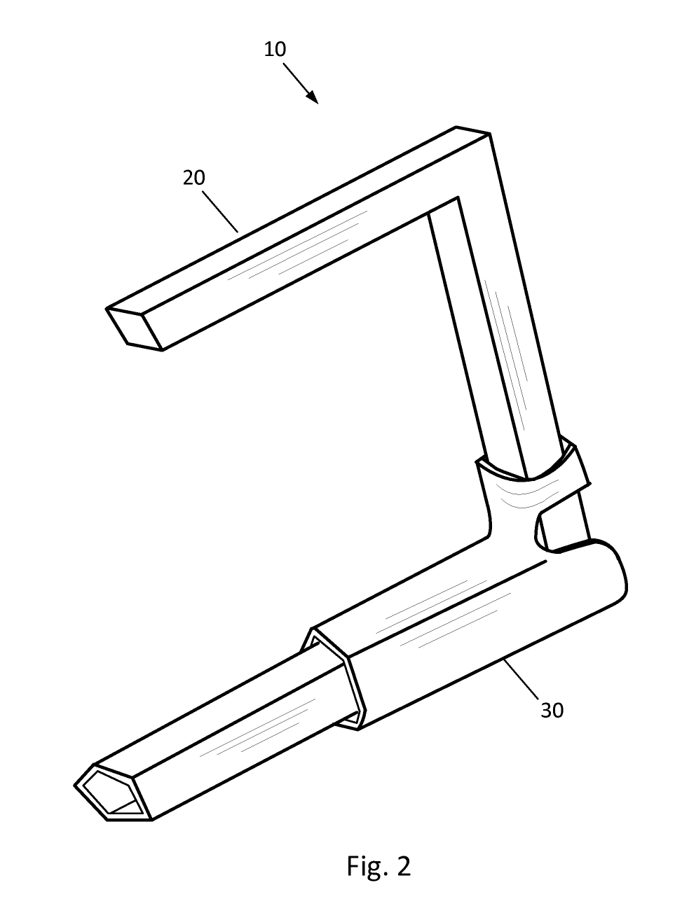

An operative laryngoscope with dental guard 10 illustrated in FIG. 1 has been inserted into the larynx region of a human patient 15 through the patient's oral cavity. The operative laryngoscope with dental guard 10 provides access for medical instruments to be inserted through the operative laryngoscope 20 but also provides the novel feature of a dental guard encompassing the entire outer surface of the rigid walled portion of the operative laryngoscope 20 positioned in the oral cavity near the teeth. FIG. 2 illustrates the operative laryngoscope with dental guard 10 not inserted into a patient 15. The operative laryngoscope 20 is fitted with a removable dental guard 30 which comprises a sheath made of resilient material, the sheath having a proximal end and a distal end. The dental guard 30 is designed to be of sufficient size to cover all outer wall surfaces of the portions of the operative laryngoscope 20 that remain near the patient's teeth once the operative laryngoscope 20 is inserted into the patient 15. The dental guard 30 is made of a resilient material (known medical/surgical grade resilient material) to minimize the risk of dental damage to the patient 15 when there is contact between the teeth and the operative laryngoscope 20. In one embodiment of the invention, the resilient material is silicone rubber. Other resilient and elastomeric materials are also suitable. In at least one embodiment, the dental guard 30 is made of flame retardant, resilient material to avoid combustion in a potentially oxygen rich environment particularly in the presence of laser based surgical instruments. The dental guard 30 cushions the teeth when they are used as a fulcrum 12 during manipulation of the operative laryngoscope 20 in one embodiment of the invention. In at least one embodiment of the invention, the outer surfaces of the dental guard 30 which may contact dental surfaces are coated or covered with a thin layer of less resilient material 40 (approximately 1-2 mm thick plastic material in one embodiment such as known medical grade plastics, polycarbonate, acrylic, nylon, etc.) compared to the main body of the dental guard 30 to minimize friction between the outer surface of the dental guard 30 and any dental surfaces thereby facilitating axial and lateral traversing of the operative laryngoscope 20 during a medical procedure while still protecting dental surfaces of the patient 15. The dental guard 30 may be reusable in one embodiment and is a one-time use product in another embodiment. The dental guards 30 which are reusable are typically constructed of autoclavable material.

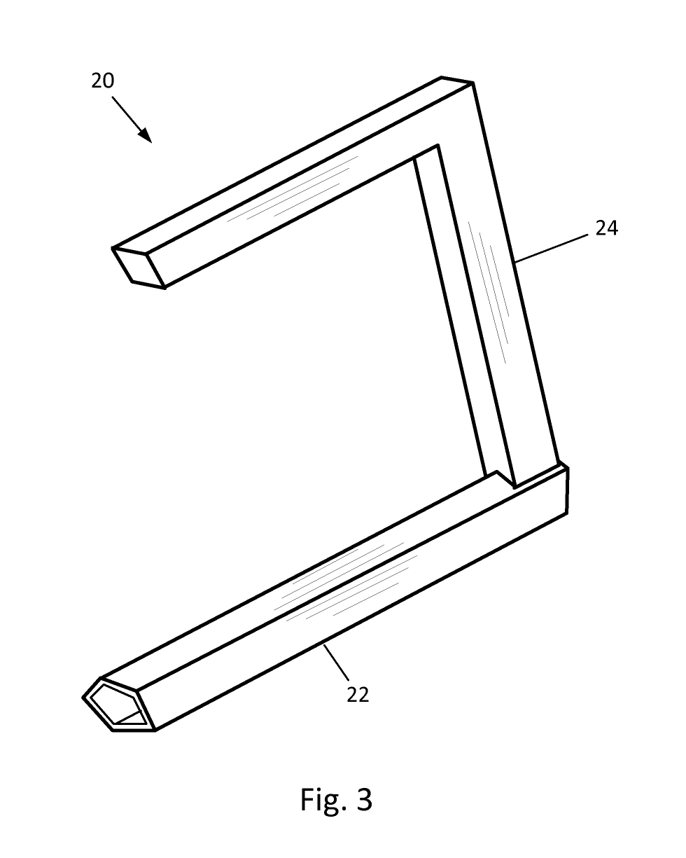

A conventional design of an operative laryngoscope 20 is illustrated in FIG. 3. It is understood that the operative laryngoscope design shown is meant to be exemplary and that any operative laryngoscope design known or used in the art may be substituted for the illustrated design and still fall within the scope of the instant invention. The operative laryngoscope 20 comprises a hollow tube section 22 and a handle section 24. The hollow tube section 22 permits access of medical instruments into the patient's larynx region when the operative laryngoscope 20 is inserted into the patient 15. The handle section 24 permits manual manipulation of the position of operative laryngoscope 20 to facilitate medical procedures performed through the hollow tube section 22. It is at least the manual manipulation of the operative laryngoscope 20 that presents a risk of dental damage to the patient's teeth if a dental guard 30 is not properly positioned and affixed to the operative laryngoscope 20. The resilient properties of the material(s) comprising the dental guard 30 provide a cushioning effect of the dental guard 30 and reduce mechanical point loading of the laryngoscope 20 on dental surfaces thereby reducing the chance of damaging teeth or gum tissue when the laryngoscope is manipulated within the oral cavity of a patient 15.

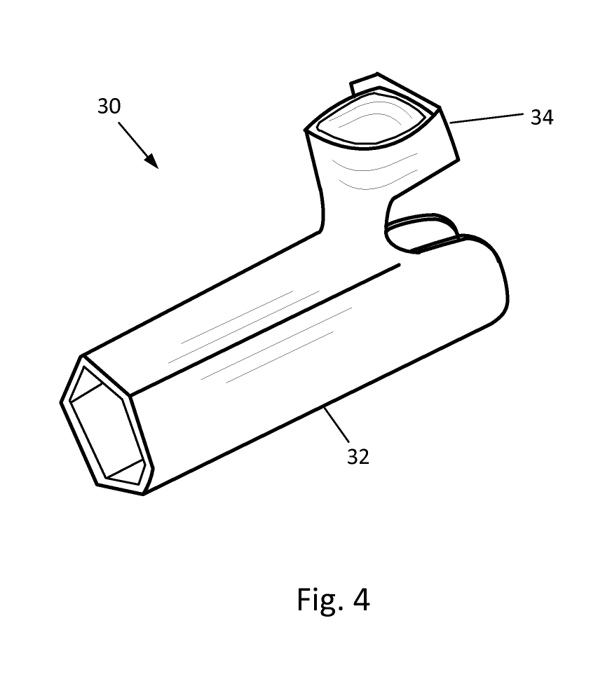

FIG. 4 illustrates the dental guard 30 with the anchor strap 34 in a fastened position without the presence of the operative laryngoscope 20. In one embodiment, the dental guard sheath 32 is manufactured with a continuous material incorporating the anchor strap 34 near the distal end of the dental guard 30.

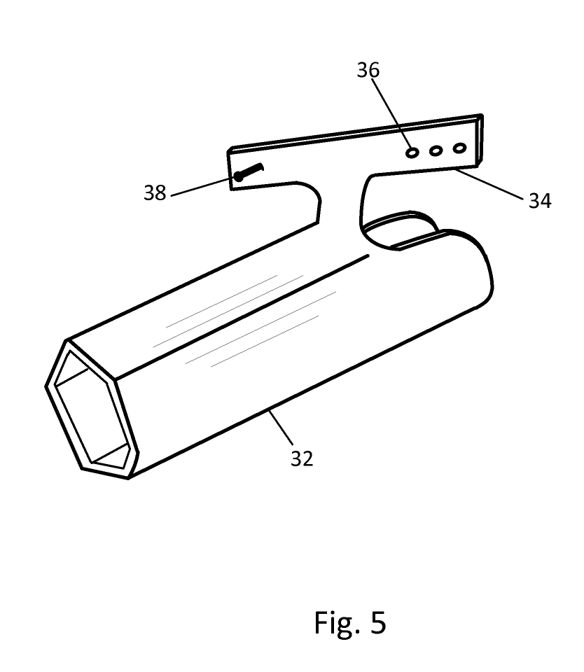

FIG. 5 illustrates the dental guard 30 with the anchor strap 34 in an unfastened position. In one embodiment, the anchor strap 34 is fastened around the handle section 24 of the operative laryngoscope 20 using a hole 36 and post 38 connection. In another embodiment, a hook and loop fastening system (not shown) is used to secure the anchor strap around the handle section 24 for one-time use dental guards. Other known strap fastening systems may be used in other embodiments.

To install the dental guard 30 on the operative laryngoscope 20, the hollow tube section 22 of the scope is inserted through the distal end of the dental guard 30 until the anchor strap 34 can be secured around the handle section 24 of the operative laryngoscope 20. Typically, the end of the laryngoscope that is intended to be positioned in the larynx region of the patient 15, extends beyond the proximal end of the dental guard 30 when the dental guard 30 is in the final position as shown in FIG. 2. The operative laryngoscope 20 with the dental guard 30 affixed is considered to be a guarded operative laryngoscope and is then inserted into the patient 15. After the laryngoscopy procedure is completed, the dental guard 30 can be removed from the operative laryngoscope 20 by unfastening the anchor strap 34 and sliding off the dental guard 30 from the operative laryngoscope 20.

It is understood that the dental guard described herein is for operative (or operating) laryngoscopes used for surgical and observation procedures performed in the larynx region of a human patient where the operative laryngoscope often needs to be manipulated omni-directionally once inserted into the patient to provide surgical access to targeted anatomy regions in the larynx. Thus, the dental guard described herein is not intended for use with intubating laryngoscopes used for tracheal intubation of human patients which are not typically used for passage of surgical instruments into the patient during a laryngoscopy procedure.

The various embodiments described herein are merely descriptive of the present invention and are in no way intended to limit the scope of the invention. Modifications of the present invention will become obvious to those having skill in the art in light of the detailed description herein, and such modifications are intended to fall within the scope of the appended claims.

I claim:

1. A dental guard for a operative laryngoscope comprising:
a sheath made from resilient material with a distal end and a proximal end;
the operative laryngoscope inserted through the distal end of the sheath; and
an anchor strap near the distal end of the sheath secures the sheath to the operative laryngoscope.

2. The dental guard of claim 1 wherein the resilient material is also flame retardant.

3. The dental guard of claim 1 wherein the anchor strap is secured with a hole and post connection.

4. The dental guard of claim 1 wherein the anchor strap is secured by a hook and loop fastener.

5. The dental guard of claim 1 wherein the resilient material comprises silicone rubber.

6. The dental guard of claim 1 wherein at least a portion of the sheath made from resilient material that contacts dental surfaces is covered with a thin layer of less resilient material.

7. The less resilient material of claim 6 comprises a medical grade plastic.

8. An operative laryngoscope with a removable dental guard comprising:
an operative laryngoscope having rigid walls and a portion which extends through the oral cavity of a human patient providing access for surgical instruments in the larynx region of the patient;
at least the portion of the operative laryngoscope which extends through the oral cavity of the patient is enveloped with a removable dental guard to protect the dental surfaces of the patient; and
the dental guard is comprised of resilient material to provide a cushion between the rigid walls of the operative laryngoscope and dental surfaces of the patient during a laryngoscopy procedure on the patient.

9. The dental guard of claim 8 wherein the resilient material is also flame retardant.

10. The dental guard of claim 8 wherein the anchor strap is secured with a hole and post connection.

11. The dental guard of claim 8 wherein the anchor strap is secured by a hook and loop fastener.

12. The dental guard of claim 8 wherein the resilient material comprises silicone rubber.

13. The dental guard of claim 8 wherein at least a portion of the sheath made from resilient material that contacts dental surfaces is covered with a thin layer of less resilient material.

14. The less resilient material of claim 13 comprises a medical grade plastic.

15. A method for protecting dental surfaces of a patient undergoing a laryngoscopy procedure comprising the steps of:
sliding a portion of an operative laryngoscope inside a removable dental guard comprised of a sheath made of resilient material;
fastening the dental guard to the operative laryngoscope to yield a guarded operative laryngoscope;
inserting the guarded operative laryngoscope through the oral cavity of a patient; and
positioning the guarded operative laryngoscope against at least one dental surface of the patient during the laryngoscopy procedure.

16. The dental guard of claim 15 wherein the resilient material is also flame retardant.

17. The dental guard of claim 15 wherein an anchor strap secures the sheath with a hole and post connection.

18. The dental guard of claim 15 wherein an anchor strap secures the sheath by a hook and loop fastener.

19. The dental guard of claim 15 wherein the resilient material comprises silicone rubber.3

20. The dental guard of claim 15 wherein at least a portion of the sheath made from resilient material that contacts dental surfaces is covered with a thin layer of less resilient material.
